(19) United States
(12) Patent Application Publication
(10) Pub. No.: US 2012/0294899 A1
(43) Pub. Date: Nov. 22, 2012

(54) Pharmaceutical composition and methods of treating and preventing the diseases caused by HIV or associated with HIV

(76) Inventors: Oleg IIiich Epshtein, Moscow (RU); Sergey Alexandrovich Tarasov, Golitsyno (RU)

(21) Appl. No.: 13/135,883

(22) Filed: Jul. 15, 2011

(30) Foreign Application Priority Data
Aug. 6, 2010 (RU) 2010133048

(51) Int. Cl. A61K 39/42; A61K 9/00; A61P 31/18

(52) U.S. Cl. 424/400; 424/160.1; 424/148.1; 424/158.1

(57) ABSTRACT

The present invention relates to a pharmaceutical composition, comprising an activated-potentiated form of an antibody to HIV protein, and method of treating and preventing the diseases caused by HIV or associated with HIV, including AIDS.

### FIELD

The present invention relates to a pharmaceutical composition and method of treating and preventing the diseases caused by HIV or associated with HIV.

### BACKGROUND

The invention relates to the area of medicine and may be used for the treatment and preventing the diseases caused by HIV or associated with HIV, including AIDS.

Treatment of viral diseases based on ultra-low doses of antibodies to interferon is known in the art (RU 2192888 C1, A61K39/395, Nov. 20, 2002). However, the given medical product can be not effective enough for treatment of the diseases associated with HIV.

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of treating diseases of viral etiology. See U.S. Pat. No. 7,572,441, which is incorporated herein by reference in its entirety.

The present invention is directed to a pharmaceutical composition and methods of its use in treatment and preventing of the diseases caused by HIV or associated with HIV, including AIDS.

The solution to the existing problem is presented in form of a pharmaceutical composition for treatment and prophylaxis (prevention) of diseases or conditions caused by HIV or associated with HIV, which comprises activated-potentiated form of antibodies to HIV protein.

### SUMMARY

In one aspect, the invention provides a pharmaceutical composition comprising an activated-potentiated form of an antibody to HIV protein. In an embodiment, the pharmaceutical composition further comprises a solid carrier, wherein said activated-potentiated form of an antibody to HIV protein is impregnated onto said solid carrier. In a variant, the pharmaceutical composition is in the form of a tablet.

In one variant of this aspect of the invention, HIV protein is HIV Gag-Pol polyprotein.

In another variant of this aspect of the invention, HIV protein is HIV enzyme. Preferably, HIV enzyme is HIV protease. It is also contemplated, that HIV enzyme is HIV integrase (HIV endonuclease). It is also contemplated that HIV enzyme is HIV reverse transcriptase.

In another variant of this aspect of the invention, HIV protein is HIV capsid protein P24 (P24 protein). It is also contemplated, that HIV protein is HIV matrix protein P17 (P17 protein).

Preferably, the pharmaceutical composition including said activated-potentiated form of an antibody to HIV protein is in the form of a mixture of C12, C30, and C200 homeopathic dilutions. It is specifically contemplated that said mixture of C12, C30, and C200 homeopathic dilutions is impregnated onto a solid carrier.

The activated-potentiated form of an antibody to HIV protein may be a monoclonal, polyclonal or natural antibody. It is specifically contemplated that the activated-potentiated form of an antibody to HIV protein is a polyclonal antibody. The invention provides activated-potentiated forms of antibodies to antigen(s) having sequences described in the specification and claimed in the appended claims. In a variant, the pharmaceutical composition includes activated-potentiated form of an antibody to HIV protein prepared by successive centesimal dilutions coupled with shaking of every dilution. Vertical shaking is specifically contemplated.

In another aspect, the invention provides a method of treating and preventing the diseases caused by HIV or associated with HIV, including AIDS, said method comprising administering to a patient in need thereof an activated-potentiated form of an antibody to HIV protein. Preferably, the activated-potentiated form of an antibody to HIV protein is administered in the form of pharmaceutical composition.

In an embodiment, the pharmaceutical composition is administered in the form of a solid oral dosage form which comprises a pharmaceutically acceptable carrier and said activated-potentiated form of an antibody to HIV protein impregnated onto said carrier. In a variant, said solid oral dosage form is a tablet. Variants and embodiments are provided.

In accordance with the method aspect of the invention, the pharmaceutical composition may be administered in one to two unit dosage forms, each of the dosage form being administered from once daily to four times daily. In a variant, the pharmaceutical composition is administered twice daily, each administration consisting of two oral dosage forms. In a variant, the pharmaceutical composition is administered in one to two unit dosage forms, each of the dosage forms being administered twice daily. All variants and embodiments described with respect to the composition aspect of the invention may be used with the method aspect of the invention.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well-accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated-potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science; it is associated with higher antiviral and, possibly, immunotropic action, intensification of activation of CD4 lymphocytes and enrichment of number of receptors on the surface of CD4 cells.

Thus, loss of viral load is observed as a result of repression of HIV entering the cells (exhibited as a change in functional activity of CD4 receptors through which HIV enters the cells); repression of replication of HIV inside the cells, activation of the process of transcription of mRNA of antiviral protein (protein kinase PKR, oligoadenylate synthetase, adenozime deaminase), Mx, MHC I and II protein etc.). Thus, the claimed medicinal product possesses high preventive effectiveness with respect to HIV, preventing infection of the cells by HIV and its endocellular replication. It can be used either for effective treatment or for preventive measures of chronic viral diseases, including secondary prevention of HIV infection.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated’ form of the antibodies of the present invention is not attributable to the initial molecular form of the antibody. Preferred is the “activated-potentiated” form of antibody in liquid or solid form in which the concentration of the molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides a pharmaceutical composition that includes activated-potentiated form of antibodies to HIV protein, prepared according to the homeopathic technology of potentiation by repeated, consistent dilution and intermediate external action of shaking as described in more detail herein below. The pharmaceutical composition of the invention is particularly useful in the treatment and prophylaxis of the diseases caused by HIV or associated with HIV, including AIDS. As shown in the Examples, the pharmaceutical composition of the invention possesses unexpected therapeutic effect, which manifest itself in particular therapeutic effectiveness in treatment of diseases caused by HIV or associated with HIV.

The pharmaceutical composition of the invention expands the arsenal of preparations available for the treatment prophylaxis of the diseases caused by HIV or associated with HIV, including AIDS.

The pharmaceutical composition in accordance with this aspect of the invention may be in the liquid form or in solid form. Activated potentiated form of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R.—2005—Vol. 14.—N 1-2. P.33-55, both incorporated herein by reference.

Monoclonal antibodies may be obtained, e.g., by means of hybridoma technology. The initial stage of the process includes immunization based on the principles already developed in the course of polyclonal antisera preparation. Further stages of work involve the production of hybrid cells generating clones of antibodies with identical specificity. Their separate isolation is performed using the same methods as in the case of polyclonal antisera preparation.

Polyclonal antibodies may be obtained via active immunization of animals. For this purpose, for example, suitable animals (e.g. rabbits) receive a series of injections of the appropriate antigen (HIV protein). The animals' immune system generates corresponding antibodies, which are collected from the animals in a known manner. This procedure enables preparation of a monospecific antibody-rich serum.

If desired, the serum containing antibodies may be purified, for example by using affine chromatography, fractionation by salt precipitation, or ion-exchange chromatography. The resulting purified, antibody-enriched serum may be used as a starting material for the preparation of the activated-potentiated form of the antibodies. The preferred concentration of the resulting initial solution of antibody in the solvent, preferably water or a water-ethyl alcohol mixture, ranges from about 0.5 to about 5.0 mg/ml.

The preferred procedure for preparing each component of the combination drug according to the present invention is the use of the mixture of three aqueous-alcohol dilutions of the primary matrix solution of antibodies diluted 100¹², 100³⁰ and 100⁵⁰ times, respectively, which is equivalent to centesimal homeopathic dilutions C12, C30, and C50 or diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the pharmaceutical composition of the invention is polyclonal, animal-raised antibody to the corresponding antigen, namely, HIV protein. To obtain the activated-potentiated form of polyclonal antibodies to HIV protein, the desired antigen may be injected as immunogen into a laboratory animal, preferably, rabbits. Polyclonal antibodies to HIV protein may be obtained using the whole molecule of HIV Gag-Pol polyprotein of the following sequence:

```
SEQ ID NO: 1
Met Gly Ala Arg Ala Ser Val Leu Ser Gly Gly Glu Leu Asp Arg
  1 5 10 15
Trp Glu Lys Ile Arg Leu Arg Pro Gly Gly Lys Lys Lys Tyr Lys
  16 20 25 30
Leu Lys His Ile Val Trp Ala Ser Arg Glu Leu Glu Arg Phe Ala
  31 35 40 45
Val Asn Pro Gly Leu Leu Glu Thr Ser Glu Gly Cys Arg Gln Ile
  46 50 55 60
Leu Gly Gln Leu Gln Pro Ser Leu Gln Thr Gly Ser Glu Glu Leu
  61 65 70 75
Arg Ser Leu Tyr Asn Thr Val Ala Thr Leu Tyr Cys Val His Gln
  76 80 85 90
Arg Ile Glu Ile Lys Asp Thr Lys Glu Ala Leu Asp Lys Ile Glu
  91 95 100 105
Glu Glu Gln Asn Lys Ser Lys Lys Lys Ala Gln Gln Ala Ala Ala
106 110 115 120
Asp Thr Gly His Ser Asn Gln Val Ser Gln Asn Tyr Pro Ile Val
121 125 130 135
Gln Asn Ile Gln Gly Gln Met Val His Gln Ala Ile Ser Pro Arg
136 140 145 150
Thr Leu Asn Ala Trp Val Lys Val Val Glu Glu Lys Ala Phe Ser
151 155 160 165
Pro Glu Val Ile Pro Met Phe Ser Ala Leu Ser Glu Gly Ala Thr
166 170 175 180
Pro Gln Asp Leu Asn Thr Met Leu Asn Thr Val Gly Gly His Gln
181 185 190 195
Ala Ala Met Gln Met Leu Lys Glu Thr Ile Asn Glu Glu Ala Ala
196 200 205 210
Glu Trp Asp Arg Val His Pro Val His Ala Gly Pro Ile Ala Pro
211 215 220 225
Gly Gln Met Arg Glu Pro Arg Gly Ser Asp Ile Ala Gly Thr Thr
226 230 235 240
Ser Thr Leu Gln Glu Gln Ile Gly Trp Met Thr Asn Asn Pro Pro
241 245 250 255
Ile Pro Val Gly Glu Ile Tyr Lys Arg Trp Ile Ile Leu Gly Leu
256 260 265 270
Asn Lys Ile Val Arg Met Tyr Ser Pro Thr Ser Ile Leu Asp Ile
271 275 280 285
Arg Gln Gly Pro Lys Glu Pro Phe Arg Asp Tyr Val Asp Arg Phe
286 290 295 300
Tyr Lys Thr Leu Arg Ala Glu Gln Ala Ser Gln Glu Val Lys Asn
301 305 310 315
Trp Met Thr Glu Thr Leu Leu Val Gln Asn Ala Asn Pro Asp Cys
346 350 355 360
Lys Thr Ile Leu Lys Ala Leu Gly Pro Ala Ala Thr Leu Glu Glu
361 365 370 375
Met Met Thr Ala Cys Gln Gly Val Gly Gly Pro Gly His Lys Ala
Arg Val Leu Ala Glu Ala Met Ser Gln Val Thr Asn Ser Ala Thr
376 380 385 390
Ile Met Met Gln Arg Gly Asn Phe Arg Asn Gln Arg Lys Ile Val
391 395 400 405
Lys Cys Phe Asn Cys Gly Lys Glu Gly His Thr Ala Arg Asn Cys
406 410 415 420
Arg Ala Pro Arg Lys Lys Gly Cys Trp Lys Cys Gly Lys Glu Gly
421 425 430 435
His Gln Met Lys Asp Cys Thr Glu Arg Gln Ala Asn Phe Leu Arg
436 440 445 450
Glu Asp Leu Ala Phe Leu Gln Gly Lys Ala Arg Glu Phe Ser Ser
451 455 460 465
Glu Gln Thr Arg Ala Asn Ser Pro Thr Arg Arg Glu Leu Gln Val
466 470 475 480
Trp Gly Arg Asp Asn Asn Ser Pro Ser Glu Ala Gly Ala Asp Arg
481 485 490 495
Gln Gly Thr Val Ser Phe Asn Phe Pro Gln Val Thr Leu Trp Gln
496 500 505 510
Arg Pro Leu Val Thr Ile Lys Ile Gly Gly Gln Leu Lys Glu Ala
511 515 510 525
Leu Leu Asp Thr Gly Ala Asp Asp Thr Val Leu Glu Glu Met Ser
526 530 535 540
Leu Pro Gly Arg Trp Lys Pro Lys Met Ile Gly Gly Ile Gly Gly
541 545 550 555
Phe Ile Lys Val Arg Gln Tyr Asp Gln Ile Leu Ile Glu Ile Cys
556 560 565 570
Gly His Lys Ala Ile Gly Thr Val Leu Val Gly Pro Thr Pro Val
571 575 580 585
Asn Ile Ile Gly Arg Asn Leu Leu Thr Gln Ile Gly Cys Thr Leu
586 590 595 600
Asn Phe Pro Ile Ser Pro Ile Glu Thr Val Pro Val Lys Leu Lys
601 605 610 615
Pro Gly Met Asp Gly Pro Lys Val Lys Gln Trp Pro Leu Thr Glu
616 620 625 630
Glu Lys Ile Lys Ala Leu Val Glu Ile Cys Thr Glu Met Glu Lys
631 635 640 645
Glu Gly Lys Ile Ser Lys Ile Gly Pro Glu Asn Pro Tyr Asn Thr
646 650 655 660
Pro Val Phe Ala Ile Lys Lys Lys Asp Ser Thr Lys Trp Arg Lys
661 665 670 675
Leu Val Asp Phe Arg Glu Leu Asn Lys Arg Thr Gln Asp Phe Trp
676 680 685 690
Glu Val Gln Leu Gly Ile Pro His Pro Ala Gly Leu Lys Lys Lys
691 695 700 705
Lys Ser Val Thr Val Leu Asp Val Gly Asp Ala Tyr Phe Ser Val
706 710 715 720
Pro Leu Asp Glu Asp Phe Arg Lys Tyr Thr Ala Phe Thr Ile Pro
721 725 730 735
Ser Ile Asn Asn Glu Thr Pro Gly Ile Arg Tyr Gln Tyr Asn Val
736 740 745 750
Leu Pro Gln Gly Trp Lys Gly Ser Pro Ala Ile Phe Gln Ser Ser
751 755 760 765
Met Thr Lys Ile Leu Glu Pro Phe Arg Lys Gln Asn Pro Asp Ile
766 770 775 780
Val Ile Tyr Gln Tyr Met Asp Asp Leu Tyr Val Gly Ser Asp Leu
781 785 790 795
Glu Ile Gly Gln His Arg Thr Lys Ile Glu Glu Leu Arg Gln His
781 785 790 795
Leu Leu Arg Trp Gly Leu Thr Thr Pro Asp Lys Lys His Gln Lys
796 800 805 810
Glu Pro Pro Phe Leu Trp Met Gly Tyr Glu Leu His Pro Asp Lys
811 815 820 825
Trp Thr Val Gln Pro Ile Val Leu Pro Glu Lys Asp Ser Trp Thr
826 830 835 840
Val Asn Asp Ile Gln Lys Leu Val Gly Lys Leu Asn Trp Ala Ser
841 845 850 855
Gln Ile Tyr Pro Gly Ile Lys Val Arg Gln Leu Cys Lys Leu Leu
856 860 865 870
Arg Gly Thr Lys Ala Leu Thr Glu Val Ile Pro Leu Thr Glu Glu
871 875 880 885
Ala Glu Leu Glu Leu Ala Glu Asn Arg Glu Ile Leu Lys Glu Pro
886 890 895 900
Val His Gly Val Tyr Tyr Asp Pro Ser Lys Asp Leu Ile Ala Glu
901 905 910 915
Ile Gln Lys Gln Gly Gln Gly Gln Trp Thr Tyr Gln Ile Tyr Gln
916 920 925 930
Glu Pro Phe Lys Asn Leu Lys Thr Gly Lys Tyr Ala Arg Met Arg
931 935 940 945
Gly Ala His Thr Asn Asp Val Lys Gln Leu Thr Glu Ala Val Gln
946 950 955 960
Lys Ile Thr Thr Glu Ser Ile Val Ile Trp Gly Lys Thr Pro Lys
961 965 970 975
Phe Lys Leu Pro Ile Gln Lys Glu Thr Trp Glu Thr Trp Trp Thr
976 980 985 990
Glu Tyr Trp Gln Ala Thr Trp Ile Pro Glu Trp Glu Phe Val Asn
991 995 1000 1005
Thr Pro Pro Leu Val Lys Leu Trp Tyr Gln Leu Glu Lys Glu Pro
1006 1010 1015 1020
Ile Val Gly Ala Glu Thr Phe Tyr Val Asp Gly Ala Ala Asn Arg
1021 1025 1030 1035
Glu Thr Lys Leu Gly Lys Ala Gly Tyr Val Thr Asn Arg Gly Arg
1036 1040 1045 1050
Gln Lys Val Val Thr Leu Thr Asp Thr Thr Asn Gln Lys Thr Glu
1051 1055 1060 1065
Leu Gln Ala Ile Tyr Leu Ala Leu Gln Asp Ser Gly Leu Glu Val
1066 1070 1075 1080
Asn Ile Val Thr Asp Ser Gln Tyr Ala Leu Gly Ile Ile Gln Ala
1081 1085 1090 1095
Gln Pro Asp Gln Ser Glu Ser Glu Leu Val Asn Gln Ile Ile Glu
1096 1100 1105 1110
Gln Leu Ile Lys Lys Glu Lys Val Tyr Leu Ala Trp Val Pro Ala
1111 1115 1120 1125
His Lys Gly Ile Gly Gly Asn Glu Gln Val Asp Lys Leu Val Ser
1126 1130 1135 1140
Ala Gly Ile Arg Lys Val Leu Phe Leu Asp Gly Ile Asp Lys Ala
1141 1145 1150 1155
Gln Asp Glu His Glu Lys Tyr His Ser Asn Trp Arg Ala Met Ala
1156 1160 1165 1170
Ser Asp Phe Asn Leu Pro Pro Val Val Ala Lys Glu Ile Val Ala
1171 1175 1180 1185
Ser Cys Asp Lys Cys Gln Leu Lys Gly Glu Ala Met His Gly Gln
1186 1190 1195 1200
Val Asp Cys Ser Pro Gly Ile Trp Gln Leu Asp Cys Thr His Leu
1201 1205 1210 1215
Glu Gly Lys Val Ile Leu Val Ala Val His Val Ala Ser Gly Tyr
1216 1220 1225 1230
Ile Glu Ala Glu Val Ile Pro Ala Glu Thr Gly Gln Glu Thr Ala
1231 1235 1240 1245
Tyr Phe Leu Leu Lys Leu Ala Gly Arg Trp Pro Val Lys Thr Ile
1246 1250 1255 1260
His Thr Asp Asn Gly Ser Asn Phe Thr Gly Ala Thr Val Arg Ala
1261 1265 1270 1275
Ala Cys Trp Trp Ala Gly Ile Lys Gln Glu Phe Gly Ile Pro Tyr
1276 1280 1285 1290
Asn Pro Gln Ser Gln Gly Val Val Glu Ser Met Asn Lys Glu Leu
1291 1295 1300 1305
Lys Lys Ile Ile Gly Gln Val Arg Asp Gln Ala Glu His Leu Lys
1306 1310 1315 1320
Thr Ala Val Gln Met Ala Val Phe Ile His Asn Phe Lys Arg Lys
1321 1325 1330 1335
Gly Gly Ile Gly Gly Tyr Ser Ala Gly Glu Arg Ile Val Asp Ile
1336 1340 1345 1350
Ile Ala Thr Asp Ile Gln Thr Lys Glu Leu Gln Lys Gln Ile Thr
1351 1355 1360 1365
Lys Ile Gln Asn Phe Arg Val Tyr Tyr Arg Asp Ser Arg Asn Pro
1366 1370 1375 1380
Leu Trp Lys Gly Pro Ala Lys Leu Leu Trp Lys Gly Glu Gly Ala
1381 1385 1390 1395
Val Val Ile Gln Asp Asn Ser Asp Ile Lys Val Val Pro Arg Arg
1396 1400 1405 1410
Lys Ala Lys Ile Ile Arg Asp Tyr Gly Lys Gln Met Ala Gly Asp
1411 1415 1420 1425
Asp Cys Val Ala Ser Arg Gln Asp Glu Asp.
1426 1430 1435
```

Polyclonal antibodies to HIV protein may be obtained using the molecule of Matix protein P17 (P17 protein) of the following sequence:

```
SEQ ID NO: 2
        Gly Ala Arg Ala Ser Val Leu Ser Gly Gly Glu Leu Asp Arg
          2 5 10 15
Trp Glu Lys Ile Arg Leu Arg Pro Gly Gly Lys Lys Lys Tyr Lys
  16 20 25 30
Leu Lys His Ile Val Trp Ala Ser Arg Glu Leu Glu Arg Phe Ala
  31 35 40 45
Val Asn Pro Gly Leu Leu Glu Thr Ser Glu Gly Cys Arg Gln Ile
  46 50 55 60
Leu Gly Gln Leu Gln Pro Ser Leu Gln Thr Gly Ser Glu Glu Leu
  61 65 70 75
Arg Ser Leu Tyr Asn Thr Val Ala Thr Leu Tyr Cys Val His Gln
  76 80 85 90
Arg Ile Glu Ile Lys Asp Thr Lys Glu Ala Leu Asp Lys Ile Glu
  91 95 100 105
Glu Glu Gln Asn Lys Ser Lys Lys Lys Ala Gln Gln Ala Ala Ala
106 110 115 120
Asp Thr Gly His Ser Asn Gln Val Ser Gln Asn Tyr
121 125 130 132
```

Polyclonal antibodies to HIV protein may be obtained using the molecule of Capsid protein P24 (P24 protein) of the following sequence:

```
SEQ ID NO: 3
                                                                                                Pro Ile Val
                                                                                                133 135
Gln Asn Ile Gln Gly Gln Met Val His Gln Ala Ile Ser Pro Arg
136 140 145 150
Thr Leu Asn Ala Trp Val Lys Val Val Glu Glu Lys Ala Phe Ser
151 155 160 165
Pro Glu Val Ile Pro Met Phe Ser Ala Leu Ser Glu Gly Ala Thr
166 170 175 180
Pro Gln Asp Leu Asn Thr Met Leu Asn Thr Val Gly Gly His Gln
181 185 190 195
Ala Ala Met Gln Met Leu Lys Glu Thr Ile Asn Glu Glu Ala Ala
196 200 205 210
Glu Trp Asp Arg Val His Pro Val His Ala Gly Pro Ile Ala Pro
211 215 220 225
Gly Gln Met Arg Glu Pro Arg Gly Ser Asp Ile Ala Gly Thr Thr
226 230 235 240
Ser Thr Leu Gln Glu Gln Ile Gly Trp Met Thr Asn Asn Pro Pro
241 245 250 255
Ile Pro Val Gly Glu Ile Tyr Lys Arg Trp Ile Ile Leu Gly Leu
256 260 265 270
Asn Lys Ile Val Arg Met Tyr Ser Pro Thr Ser Ile Leu Asp Ile
271 275 280 285
Arg Gln Gly Pro Lys Glu Pro Phe Arg Asp Tyr Val Asp Arg Phe
286 290 295 300
Tyr Lys Thr Leu Arg Ala Glu Gln Ala Ser Gln Glu Val Lys Asn
301 305 310 315
Trp Met Thr Glu Thr Leu Leu Val Gln Asn Ala Asn Pro Asp Cys
346 350 355 360
Lys Thr Ile
361 363
```

Polyclonal antibodies to HIV protein may be obtained using the molecule of HIV protease of the following sequence:

```
SEQ ID NO: 4
                                                                Ser Glu Ala Gly Ala Asp Arg
                                                                489 490 495
Gln Gly Thr Val Ser Phe Asn Phe Pro Gln Val Thr Leu Trp Gln
496 500 505 510
Arg Pro Leu Val Thr Ile Lys Ile Gly Gly Gln Leu Lys Glu Ala
511 515 510 525
Leu Leu Asp Thr Gly Ala Asp Asp Thr Val Leu Glu Glu Met Ser
526 530 535 540
Leu Pro Gly Arg Trp Lys Pro Lys Met Ile Gly Gly Ile Gly Gly
541 545 550 555
Phe Ile Lys Val Arg Gln Tyr Asp Gln Ile Leu Ile Glu Ile Cys
556 560 565 570
Gly His Lys Ala Ile Gly Thr Val Leu Val Gly Pro Thr Pro Val
571 575 580 585
Asn Ile
586 587
```

Polyclonal antibodies to HIV protein may be obtained using the molecule of HIV integrase (HIV endonuclease) of the following sequence:

```
SEQ ID NO: 5
                                                        Phe Leu Asp Gly Ile Asp Lys Ala
                                                      1148 1150 1155
Gln Asp Glu His Glu Lys Tyr His Ser Asn Trp Arg Ala Met Ala
1156 1160 1165 1170
Ser Asp Phe Asn Leu Pro Pro Val Val Ala Lys Glu Ile Val Ala
1171 1175 1180 1185
Ser Cys Asp Lys Cys Gln Leu Lys Gly Glu Ala Met His Gly Gln
1186 1190 1195 1200
Val Asp Cys Ser Pro Gly Ile Trp Gln Leu Asp Cys Thr His Leu
1201 1205 1210 1215
Glu Gly Lys Val Ile Leu Val Ala Val His Val Ala Ser Gly Tyr
1216 1220 1225 1230
Ile Glu Ala Glu Val Ile Pro Ala Glu Thr Gly Gln Glu Thr Ala
1231 1235 1240 1245
Tyr Phe Leu Leu Lys Leu Ala Gly Arg Trp Pro Val Lys Thr Ile
1246 1250 1255 1260
His Thr Asp Asn Gly Ser Asn Phe Thr Gly Ala Thr Val Arg Ala
1261 1265 1270 1275
Ala Cys Trp Trp Ala Gly Ile Lys Gln Glu Phe Gly Ile Pro Tyr
1276 1280 1285 1290
Asn Pro Gln Ser Gln Gly Val Val Glu Ser Met Asn Lys Glu Leu
1291 1295 1300 1305
Lys Lys Ile Ile Gly Gln Val Arg Asp Gln Ala Glu His Leu Lys
1306 1310 1315 1320
Thr Ala Val Gln Met Ala Val Phe Ile His Asn Phe Lys Arg Lys
1321 1325 1330 1335
Gly Gly Ile Gly Gly Tyr Ser Ala Gly Glu Arg Ile Val Asp Ile
1336 1340 1345 1350
Ile Ala Thr Asp Ile Gln Thr Lys Glu Leu Gln Lys Gln Ile Thr
1351 1355 1360 1365
Lys Ile Gln Asn Phe Arg Val Tyr Tyr Arg Asp Ser Arg Asn Pro
1366 1370 1375 1380
Leu Trp Lys Gly Pro Ala Lys Leu Leu Trp Lys Gly Glu Gly Ala
1381 1385 1390 1395
Val Val Ile Gln Asp Asn Ser Asp Ile Lys Val Val Pro Arg Arg
1396 1400 1405 1410
Lys Ala Lys Ile Ile Arg Asp Tyr Gly Lys Gln Met Ala Gly Asp
1411 1415 1420 1425
Asp Cys Val Ala Ser Arg Gln Asp Glu Asp
1426 1430 1435
```

Polyclonal antibodies to HIV protein may be obtained using the molecule of HIV reverse transcriptase of the following sequence:

```
SEQ ID NO: 6
                Ile Gly Arg Asn Leu Leu Thr Gln Ile Gly Cys Thr Leu
                588 590 595 600
Asn Phe Pro Ile Ser Pro Ile Glu Thr Val Pro Val Lys Leu Lys
601 605 610 615
Pro Gly Met Asp Gly Pro Lys Val Lys Gln Trp Pro Leu Thr Glu
616 620 625 630
Glu Lys Ile Lys Ala Leu Val Glu Ile Cys Thr Glu Met Glu Lys
631 635 640 645
Glu Gly Lys Ile Ser Lys Ile Gly Pro Glu Asn Pro Tyr Asn Thr
646 650 655 660
Pro Val Phe Ala Ile Lys Lys Lys Asp Ser Thr Lys Trp Arg Lys
661 665 670 675
Leu Val Asp Phe Arg Glu Leu Asn Lys Arg Thr Gln Asp Phe Trp
676 680 685 690
Glu Val Gln Leu Gly Ile Pro His Pro Ala Gly Leu Lys Lys Lys
691 695 700 705
Lys Ser Val Thr Val Leu Asp Val Gly Asp Ala Tyr Phe Ser Val
706 710 715 720
Pro Leu Asp Glu Asp Phe Arg Lys Tyr Thr Ala Phe Thr Ile Pro
721 725 730 735
Ser Ile Asn Asn Glu Thr Pro Gly Ile Arg Tyr Gln Tyr Asn Val
736 740 745 750
Leu Pro Gln Gly Trp Lys Gly Ser Pro Ala Ile Phe Gln Ser Ser
751 755 760 765
Met Thr Lys Ile Leu Glu Pro Phe Arg Lys Gln Asn Pro Asp Ile
766 770 775 780
Val Ile Tyr Gln Tyr Met Asp Asp Leu Tyr Val Gly Ser Asp Leu
781 785 790 795
Glu Ile Gly Gln His Arg Thr Lys Ile Glu Glu Leu Arg Gln His
781 785 790 795
Leu Leu Arg Trp Gly Leu Thr Thr Pro Asp Lys Lys His Gln Lys
796 800 805 810
Glu Pro Pro Phe Leu Trp Met Gly Tyr Glu Leu His Pro Asp Lys
811 815 820 825
Trp Thr Val Gln Pro Ile Val Leu Pro Glu Lys Asp Ser Trp Thr
826 830 835 840
Val Asn Asp Ile Gln Lys Leu Val Gly Lys Leu Asn Trp Ala Ser
841 845 850 855
Gln Ile Tyr Pro Gly Ile Lys Val Arg Gln Leu Cys Lys Leu Leu
856 860 865 870
Arg Gly Thr Lys Ala Leu Thr Glu Val Ile Pro Leu Thr Glu Glu
871 875 880 885
Ala Glu Leu Glu Leu Ala Glu Asn Arg Glu Ile Leu Lys Glu Pro
886 890 895 900
Val His Gly Val Tyr Tyr Asp Pro Ser Lys Asp Leu Ile Ala Glu
901 905 910 915
Ile Gln Lys Gln Gly Gln Gly Gln Trp Thr Tyr Gln Ile Tyr Gln
916 920 925 930
Glu Pro Phe Lys Asn Leu Lys Thr Gly Lys Tyr Ala Arg Met Arg
931 935 940 945
Gly Ala His Thr Asn Asp Val Lys Gln Leu Thr Glu Ala Val Gln
946 950 955 960
Lys Ile Thr Thr Glu Ser Ile Val Ile Trp Gly Lys Thr Pro Lys
961 965 970 975
Phe Lys Leu Pro Ile Gln Lys Glu Thr Trp Glu Thr Trp Trp Thr
976 980 985 990
Glu Tyr Trp Gln Ala Thr Trp Ile Pro Glu Trp Glu Phe Val Asn
991 995 1000 1005
Thr Pro Pro Leu Val Lys Leu Trp Tyr Gln Leu Glu Lys Glu Pro
1006 1010 1015 1020
Ile Val Gly Ala Glu Thr Phe Tyr Val Asp Gly Ala Ala Asn Arg
1021 1025 1030 1035
Glu Thr Lys Leu Gly Lys Ala Gly Tyr Val Thr Asn Arg Gly Arg
1036 1040 1045 1050
Gln Lys Val Val Thr Leu Thr Asp Thr Thr Asn Gln Lys Thr Glu
1051 1055 1060 1065
Leu Gln Ala Ile Tyr Leu Ala Leu Gln Asp Ser Gly Leu Glu Val
1066 1070 1075 1080
Asn Ile Val Thr Asp Ser Gln Tyr Ala Leu Gly Ile Ile Gln Ala
1081 1085 1090 1095
Gln Pro Asp Gln Ser Glu Ser Glu Leu Val Asn Gln Ile Ile Glu
1096 1100 1105 1110
Gln Leu Ile Lys Lys Glu Lys Val Tyr Leu Ala Trp Val Pro Ala
1111 1115 1120 1125
His Lys Gly Ile Gly Gly Asn Glu Gln Val Asp Lys Leu Val Ser
1126 1130 1135 1140
Ala Gly Ile Arg Lys Val Leu
1141 1145 1147
```

The exemplary procedure for preparation of the starting polyclonal antibodies to HIV protein may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. or without NaN₃ at the temperature of −70° C. To separate the target antibodies to HIV protein from the antiserum, the following solid phase absorption sequence is suitable:

10 ml of the antiserum of rabbits is diluted twofold with 0.15 M NaCl, after which 6.26 g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to a DEAE-cellulose column balanced by phosphate buffer. The antibody fraction is determined by measuring the optical density of the eluate at 280 nm.

The isolated crude antibodies are purified using affine chromatography method by attaching the obtained antibodies to HIV protein located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to HIV protein is 0.5 to 5.0 mg/ml, preferably, 2.0 to 3.0 mg/ml.

The activated-potentiated form of an antibody to HIV protein may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to HIV protein with the concentration of 3.0 mg/ml is diluted in 99 parts of neutral aqueous or aqueous-alcohol solvent (preferably, 15%-ethyl alcohol) and then vertically shaked many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to gamma interferon with the concentration of 3.0 mg/ml in 99 parts of a neutral solvent in different containers, which is equivalent to the centesimal homeopathic dilution C12. Similar procedures with the relevant dilution factor are performed to obtain dilutions C30, C50 and C 200. The intermediate dilutions may be tested in a desired biological model to check activity. The preferred activated-potentiated form for the composition of the invention are a mixture of C12, C30, and C50 dilutions or C12, C30 and C200 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D20, C30, C100 or C12, C30, C50 or C12, C30, C200, etc.), the efficiency of which is determined experimentally by testing the dilution in a suitable biological model, for example, in models described in the examples herein.

In the course of potentiation and concentration decrease, the vertical shaking may be substituted for external exposure to ultrasound, electromagnetic field or any similar external impact procedure accepted in the homeopathic art.

Preferably, the pharmaceutical composition of the invention may be in the form of a liquid or in the solid unit dosage form. The preferred liquid carrier is water or water-ethyl alcohol mixture.

The solid unit dosage form of the pharmaceutical composition of the invention may be prepared by impregnating a solid, pharmaceutically acceptable carrier with the mixture of the activated potentiated form aqueous or aqueous-alcohol solutions of active component. Alternatively, the carrier may be impregnated consecutively with each requisite dilution. Both orders of impregnation are acceptable.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies HIV protein. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono-olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

To prepare the solid oral form, 100-300 μm granules of lactose are impregnated with aqueous or aqueous-alcoholic solutions of the activated-potentiated form of antibodies to HIV protein in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Within Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch-XL 400 tablet press) to form 150 to 500 mg round pills, preferably, 300 mg. After tableting, 300 mg pills are obtained that are saturated with aqueous-alcohol solution (3.0-6.0 mg/pill) of the activated-potentiated form of antibodies to HIV protein in the form of a mixture of centesimal homeopathic dilutions C12, C30, and C50 or a mixture of centesimal homeopathic dilutions C12, C30 and C200.

While the invention is not limited to any specific theory, it is believed that the activated potentiated form of the antibodies described herein do not contain the molecular form of the antibody in an amount sufficient to have biological activity attributed to such molecular form. The biological activity of the combination drug (pharmaceutical composition) of the invention is amply demonstrated in the appended examples.

Preferably, for the purpose of treatment, the combination of the invention is administered from once daily to four times daily, preferably twice daily, each administration including one or two combination unit dosage forms.

The invention is further illustrated with reference to the appended non-limiting examples.

### EXAMPLES

### Example 1

The assessment of antiretroviral activity of ultra low-dose rabbit polyclonal antibodies to HIV nucleocapsid protein p24 (P24 protein) (a mixture of homoeopathic dilutions C12+C30+C50), was carried out using human peripheral blood mononuclear cells infected with the strain HIV-1 LAI in vitro. Azidothymidine (Sigma-AZ169-100 mg, Lot 107 K1578) was used as a comparator product.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2 in RPMI1640 (DIFCO) medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin).

In order to assess antiretroviral activity the products were placed in a well 15-30 minutes after cells infection with the strain HIV-1—LAI at the dose of 100 TCID50 (50 μL inoculum of the strain HIV-1-LAI). Supernatant fluids used to assess the effect of products on the inhibition of HIV replication were also collected on day 7 after infection of cells.

Before placing in a well, which contained 150 μL of cell culture, ultra low-dose antibodies to protein p24 were diluted with RPMI1640 (DIFCO) medium at a 4-fold dilution (at a 1/4 dilution) to a final volume of 50 μL. Azidothymidine was diluted with RPMI1640 (DIFCO) medium to yield a 8 nM concentration.

The products' efficiency was established by the inhibition of HIV replication which was assessed by HIV-reverse transcriptase activity in the supernatant fluid from human peripheral blood mononuclear cells using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 1).

TABLE 1
Antiretroviral activity of ultra low-dose antibodies
to protein p24 using human peripheral blood mononuclear
cells infected with the strain HIV-1-LAI in vitro

| | | Inhibition of HIV-reverse |
|---|---|---|
| | Medium Dilution | transcriptase activity |
| | Ratio RPMI1640 | (% of control) |
| Product | (DIFCO) | Day 7 |
| Ultra low-dose | ¼ | 63 ± 17 |
| antibodies to | | |
| protein p24 | | |
| Azidothymidine | — | 58 ± 7 |
| (8 nM) | | |

Thus, this experimental model demonstrated the antiretroviral activity of ultra low-dose rabbit polyclonal antibodies to HIV nucleocapsid protein p24 (a mixture of homoeopathic dilutions C12+C30+C50).

### Example 2

### Macrophages; Reverse Transcriptase; Prevention Regimen

### List of Abbreviations:

TCID50 stands for 50% Tissue Culture Infective Dose.

The assessment of antiretroviral activity of ultra low-dose rabbit polyclonal antibodies to HIV nucleocapsid protein p24 (P24 protein) (a mixture of homoeopathic dilutions C12+C30+C50), was carried out using macrophages, obtained from human peripheral blood mononuclear cells and infected with the strain HIV-1-Ba-L in vitro. Azidothymidine (Sigma-AZ169-100 mg, Lot 107 K1578) was used as a comparator product.

Human peripheral blood macrophages were obtained from human peripheral blood mononuclear cells isolated from blood of a seronegative healthy donor by centrifugation on a Ficoll-Hypaque density gradient. Human peripheral blood mononuclear cells were grown for 3 days in RPMI1640 (DIFCO), medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin), 15 ng/mL GM-CSF (granulocytic-macrophagal colony-stimulating factor). Then cells were transferred in culture plates (150000 cells/well in a 48-well plate), grown for 7 days together with 1 ng/mL GM-CSF (granulocytic-macrophagal colony-stimulating factor) and 10 ng/mL M-CSF (macrophagal colony-stimulating factor) so that the cells completely differentiate into macrophages.

In order to assess antiretroviral activity the products were placed in a well 24 prior to after cells infection with the strain HIV-1-Ba-L at the dose of 1000 TCID50 (100 μL inoculum of the strain HIV-1-Ba-L), as well as on Day 3, 7, 10, 14, 17 after infection. Supernatant fluids used to assess the effect of products on the inhibition of HIV replication were also collected on day 3, 7, 10, 14, 17 after cells infection.

Before placing in a well, which contained 750 of cell culture, ultra low-dose antibodies to protein p24 were diluted with RPMI1640 (DIFCO) medium at a 4-fold dilution (at a 1/4 dilution) to a final volume of 250 Azidothymidine was diluted with RPMI1640 (DIFCO) medium to yield a 8 nM concentration.

The products' efficiency was established by the inhibition of HIV replication which was assessed by HIV-reverse transcriptase activity in the supernatant fluid from human peripheral blood macrophages using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 2).

TABLE 2
Antiretroviral activity of ultra low-dose antibodies
to protein p24 using human peripheral blood macrophages
infected with the strain HIV-1-Ba-L in vitro

| | Medium Dilution | Inhibition of HIV-reverse | | |
|---|---|---|---|---|
| | Ratio RPMI1640 | transcriptase activity (% of control) | | |
| Product | (DIFCO) | Day 14 | Day 17 | Day 21 |
| Ultra low-dose | ¼ | 41 ± 9 | 27 ± 2 | 27 ± 5 |
| antibodies to | | | | |
| protein p24 | | | | |
| Azidothymidine | — | 82 ± 2 | 54 ± 1 | 41 ± 1 |
| (8 nM) | | | | |

Thus, this experimental model demonstrated the antiretroviral activity of ultra low-dose rabbit polyclonal antibodies to HIV nucleocapsid protein p24 (a mixture of homoeopathic dilutions C12+C30+C50).

### Example 3

The assessment of antiretroviral activity of ultra low-dose rabbit polyclonal antibodies to HIV-1 protease (a mixture of homoeopathic dilutions C12+C30+C50) (hereinafter referred to as “ultra low-dose antibodies to HIV-1 protease)), was carried out using human peripheral blood mononuclear cells infected with the strain HIV-1 LAI in vitro. Azidothymidine (Sigma-AZ169-100 mg, Lot 107 K1578) was used as a comparator product).

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2 in RPMI1640 (DIFCO) medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin).

In order to assess antiretroviral activity the products were placed in a well 15-30 minutes after cells infection with the strain HIV-1-LAI at the dose of 100 TCID50 (50 μL inoculum of the strain HIV-1-LAI). Supernatant fluids used to assess the effect of products on the inhibition of HIV replication were also collected on day 7 after infection of cells.

Before placing in a well, which contained 150 μL of cell culture, ultra low-dose antibodies to HIV-1 protease were diluted with RPMI1640 (DIFCO) medium at a 4-fold dilution (at a 1/4 dilution) to a final volume of 50 μL. Azidothymidine was diluted with RPMI1640 (DIEGO) medium to yield a 8 nM concentration.

The products' efficiency was established by the inhibition of HIV replication which was assessed by HIV-reverse transcriptase activity in the supernatant fluid from human peripheral blood mononuclear cells using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 3).

TABLE 3
Antiretroviral activity of ultra low-dose antibodies to
HIV-1 protease using human peripheral blood mononuclear
cells infected with the strain HIV-1-LAI in vitro

| | | Inhibition of HIV-reverse |
|---|---|---|
| | Medium Dilution | transcriptase activity |
| | Ratio RPMI1640 | (% of control) |
| Product | (DIFCO) | Day 7 |
| Ultra low-dose | ¼ | 60 ± 4 |
| antibodies to | | |
| HIV-1 protease | | |
| Azidothymidine | — | 58 ± 7 |
| (8 nM) | | |

Thus, this experimental model demonstrated the antiretroviral activity of ultra low-dose rabbit polyclonal antibodies to HIV-1 protease (a mixture of homoeopathic dilutions C12+C3O+C50).

### Example 4

### Macrophages; Reverse Transcriptase; Prevention Regimen

### List of Abbreviations:

TCID50 stands for 50% Tissue Culture Infective Dose.

The assessment of antiretroviral activity of ultra low-dose rabbit polyclonal antibodies to HIV-1 protease (a mixture of homoeopathic dilutions C12+C30+C50) (hereinafter referred to as “ultra low-dose antibodies to HIV-1 protease)), was carried out using macrophages, obtained from human peripheral blood mononuclear cells and infected with the strain HIV-1-Ba-L in vitro. Azidothymidine (Sigma-AZ169-100 mg, Lot 107 K1578) was used as a comparator product.

Human peripheral blood macrophages were obtained from human peripheral blood mononuclear cells isolated from blood of a seronegative healthy donor by centrifugation on a Ficoll-Hypaque density gradient. Human peripheral blood mononuclear cells were grown for 3 days in RPMI1640 (DIFCO), medium supplemented with 10% fetal calf serum (the complement was removed by heating for 45 minutes at 56° C.), 1% antibiotic solution (PSN Gibco containing 50 μg/mL of penicillin, 50 μg/mL of streptomycin and 100 μg/mL of neomycin), 15 ng/mL GM-CSF (granulocytic-macrophagal colony-stimulating factor). Then cells were transferred in culture plates (150000 cells/well in a 48-well plate), grown for 7 days together with 1 ng/mL GM-CSF (granulocytic-macrophagal colony-stimulating factor) and 10 ng/mL M-CSF (macrophagal colony-stimulating factor) so that the cells completely differentiate into macrophages.

In order to assess antiretroviral activity the products were placed in a well 24 prior to after cells infection with the strain HIV-1-Ba-L at the dose of 1000 TCID50 (100 μL inoculum of the strain HIV-1-Ba-L), as well as on Day 3, 7, 10, 14, 17 after infection. Supernatant fluids used to assess the effect of products on the inhibition of HIV replication were also collected on day 3, 7, 10, 14, 17 after cells infection.

Before placing in a well, which contained 750 of cell culture, ultra low-dose antibodies to HIV-1 protease were diluted with RPMI1640 (DIFCO) medium at a 4-fold dilution (at a ¼ dilution) to a final volume of 250 Azidothymidine was diluted with RPMI1640 (DIFCO) medium to yield a 8 nM concentration.

The products' efficiency was established by the inhibition of HIV replication which was assessed by HIV-reverse transcriptase activity in the supernatant fluid from human peripheral blood macrophages using the HIV RT RetroSys kit made by INNOVAGEN (Lot 10-059C). The supernatant fluid of cells, to which test products or azidothymidine were not inoculated, was used as control to calculate the percentage of inhibition of HIV replication (see Table 4).

TABLE 4
Antiretroviral activity of ultra low-dose antibodies
to HIV-1 protease using human peripheral blood macrophages
infected with the strain HIV-1-Ba-L in vitro

| | Medium Dilution | Inhibition of HIV-reverse | | |
|---|---|---|---|---|
| | Ratio RPMI1640 | transcriptase activity (% of control) | | |
| Product | (DIFCO) | Day 14 | Day 17 | Day 21 |
| Ultra low-dose | ¼ | 70 ± 8 | 53 ± 3 | 34 ± 4 |
| antibodies to | | | | |
| HIV-1 protease | | | | |
| Azidothymidine | — | 82 ± 2 | 54 ± 1 | 41 ± 1 |
| (8 nM) | | | | |

Thus, this experimental model demonstrated the antiretroviral activity of ultra low-dose rabbit polyclonal antibodies to HIV-1 protease (a mixture of homoeopathic dilutions C12+C30+C50).

What is claimed is:

1. A pharmaceutical composition comprising an activated-potentiated form of an antibody to HIV protein.

2. The pharmaceutical composition of claim 1, wherein HIV protein is HIV Gag-Pol polyprotein.

3. The pharmaceutical composition of claim 1, wherein HIV protein is HIV enzyme.

4. The pharmaceutical composition of claim 3, wherein HIV enzyme is HIV protease.

5. The pharmaceutical composition of claim 3, wherein HIV enzyme is HIV integrase (HIV endonuclease).

6. The pharmaceutical composition of claim 3, wherein HIV enzyme is HIV reverse transcriptase.

7. The pharmaceutical composition of claim 1, wherein HIV protein is HIV capsid protein P24.

8. The pharmaceutical composition of claim 1, wherein HIV protein is matrix protein P17.

9. The pharmaceutical composition of claim 1, wherein the activated-potentiated form of an antibody to HIV protein is in the form of a mixture of C12, C30, and C50 homeopathic dilutions impregnated onto a solid carrier.

10. The pharmaceutical composition of claim 1, wherein the activated-potentiated form of an antibody to HIV protein is in the form of a mixture of C12, C30, and C200 homeopathic dilutions impregnated onto a solid carrier.

11. The pharmaceutical composition of claim 1, wherein the activated-potentiated form of an antibody to HIV protein is a monoclonal, polyclonal or natural antibody.

12. The pharmaceutical composition of claim 11, wherein the activated-potentiated form of an antibody to HIV protein is a polyclonal antibody.

13. The pharmaceutical composition of claim 1, wherein the activated-potentiated form of an antibody to HIV protein is prepared by successive centesimal dilutions coupled with shaking of every dilution.

14. A method of treating and preventing the diseases caused by HIV or associated with HIV, said method comprising administering to a patient in need thereof an activated-potentiated form of an antibody to HIV protein.

15. A method of claim 14, wherein said diseases caused by HIV or associated with HIV is AIDS.

16. The method of claim 14 or 15, wherein the pharmaceutical composition is administered in one to two unit dosage forms, each of the dosage form being administered from once daily to four times daily.


SEQUENCE LISTING (6 sequences)

SEQ ID NO: 1 - protein, 1435 aa, Human immunodeficiency virus 1
  SOURCE 1..1435: /mol_type=“protein” /note=“group M subtype B (isolate HXB2) ” /organism=“Human immunodeficiency virus 1”
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 2 - protein, 131 aa, Human immunodeficiency virus 1
  SOURCE 1..131: /mol_type=“protein” /note=“1 group M subtype B (isolate HXB2) ” /organism=“Human immunodeficiency virus 1”
GARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 3 - protein, 201 aa, Human immunodeficiency virus 1
  SOURCE 1..201: /mol_type=“protein” /note=“group M subtype B (isolate HXB2) ” /organism=“Human immunodeficiency virus 1”
PIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTI

SEQ ID NO: 4 - protein, 99 aa, Human immunodeficiency virus 1
  SOURCE 1..99: /mol_type=“protein” /note=“group M subtype B (isolate HXB2) ” /organism=“Human immunodeficiency virus 1”
SEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNI

SEQ ID NO: 5 - protein, 288 aa, Human immunodeficiency virus 1
  SOURCE 1..288: /mol_type=“protein” /note=“group M subtype B (isolate HXB2) ” /organism=“Human immunodeficiency virus 1”
FLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 6 - protein, 575 aa, Human immunodeficiency virus 1
  SOURCE 1..575: /mol_type=“protein” /note=“group M subtype B (isolate HXB2) ” /organism=“Human immunodeficiency virus 1”
IGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL